(19) United States
(12) Patent Application Publication
(10) Pub. No.: US 2005/0090722 A1
(43) Pub. Date: Apr. 28, 2005

(54) Method and apparatus for providing UV light to blood

(76) Inventor: Thomas Perez, Chicago, IL (US)

Correspondence Address: ORUM & ROTH, 53 W. JACKSON BLVD, CHICAGO, IL, 60604, US

(21) Appl. No.: 10/926,209

(22) Filed: Aug. 25, 2004

Related U.S. Application Data
(60) Provisional application No. 60/503,678, filed on Sep. 17, 2003.

(51) Int. Cl. A61N 1/00; A61B 5/00

(52) U.S. Cl. 600/315; 607/88

(57) ABSTRACT

Ultraviolet light is applied to a patient's blood while that blood remains in the body. A port is surgically implanted in the patient. A UV pack or a UV device is connectable to the port. The UV pack has a battery or other power supply and a UV light source. The UV light source is typically LEDs. The UV pack is connected to the port via a tube or catheter. A fiber optic strand or bundle runs through the tube to provide the UV light to the blood. Similarly, the UV device is connected to the port via a tube or catheter. A fiber optic bundle or other light guide directs the light to the blood.

This application claims the benefit of U.S. Provisional Application No. 60/503,678 filed Sep. 17, 2003.

### BACKGROUND OF THE INVENTION

1. Field of the Invention

The present invention relates to a method and apparatus for providing UV light to blood.

2. Background of the Prior Art

Ultraviolet (UV) light can be used to treat a multitude of medical problems, including for example bacterial, viral and fungal infections, poisoning, fatigue, Alzheimer's disease, allergies and asthma, rheumatic diseases and arthritis, diabetes, hepatitis, and cancer. UV light sterilizes the blood and acts as an antibiotic.

The UV light is applied either to the patient's skin or directly to the blood. If the UV light is applied to the skin it is typically provided to the patient's skin either with a wrap or lamp.

UV light is commonly used to treat jaundiced babies. Because infant's skin is thin and the blood vessels are close to the surface, UV light is typical applied to the skin when treating jaundiced babies.

Applying the UV light directly to a patient's blood supply is known as photoluminescence or UV blood illumination (UBI). UV blood illumination increases oxygen, destroys toxins and boosts the immune system.

In prior art UBI, a small amount of blood is drawn from the patient, up to about 250 cc. The body has about 5.6 L of blood. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for a method of providing UV light to a patient's entire blood supply, not just a small portion of it. There is a need for a system that is convenient for the patient, which does not require regular doctor visits. There is a need for a simple system that can be used by the patient in his home.

There is a need for a system that allows for round the clock treatments or other regular treatments such as pulsed treatment or automatic periodic treatments.

There is a need for a blood illuminator that reduces the risk of infection from removing blood. There is a need for a system that reduces the number of needle sticks a patient must endure.

There is a need for a system that allows the blood to be treated on an as needed basis, such as based on how the patient is feeling at a particular time.

### SUMMARY OF THE INVENTION

The present invention is a UV device or a portable UV pack that connects to a port in the patient's artery. A port is surgically implanted in the patient. The UV pack or device is connectable to the port.

The UV pack has a battery or other power supply and a UV light source. The UV light source is typically LEDs that emit UV-C light. The UV pack or device is connected to the port via a catheter inserted into the port or a direct connection to a connecting element on the port. Fiber optic strand run through a tube to provide the UV light to the blood.

### BRIEF DESCRIPTION OF THE DRAWINGS

FIG. 1 is a portable UV pack;

FIG. 2 is a patient with a connectable UV pack;

FIG. 3 is a cross section of a UV device;

FIG. 4a is a UV catheter for use with a UV pack or with a UV device;

FIG. 4b is a cross section of the UV catheter for use with a UV pack or with a UV device;

FIG. 5 is a fiber-optic light guide for use with a UV pack or with a UV device; and

FIGS. 6a-6e are various embodiments of the UV light bulb for with the UV pack or with the UV device.

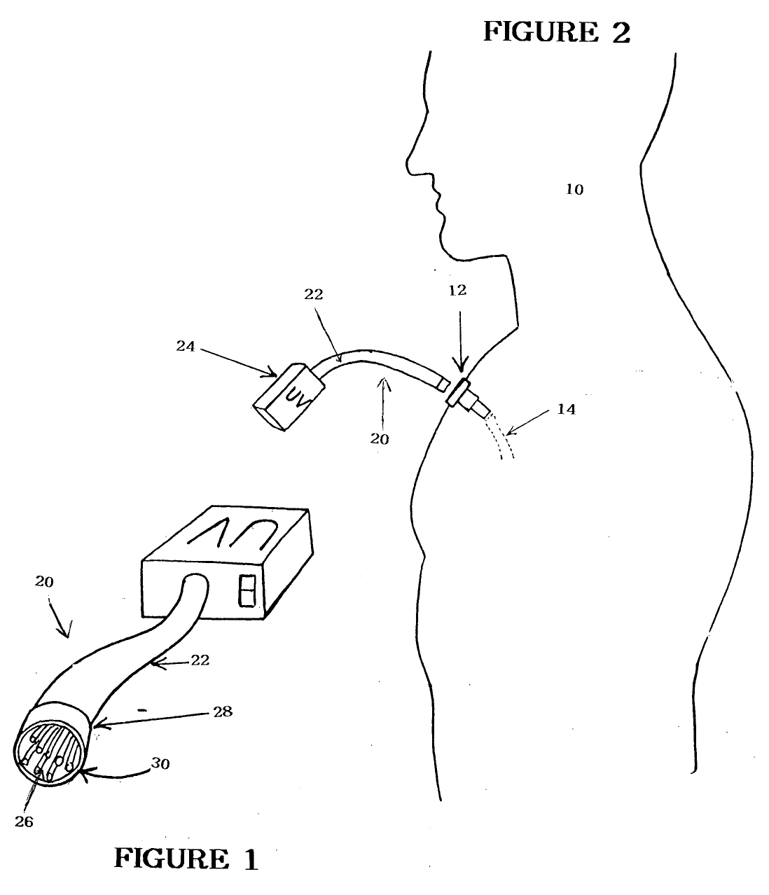

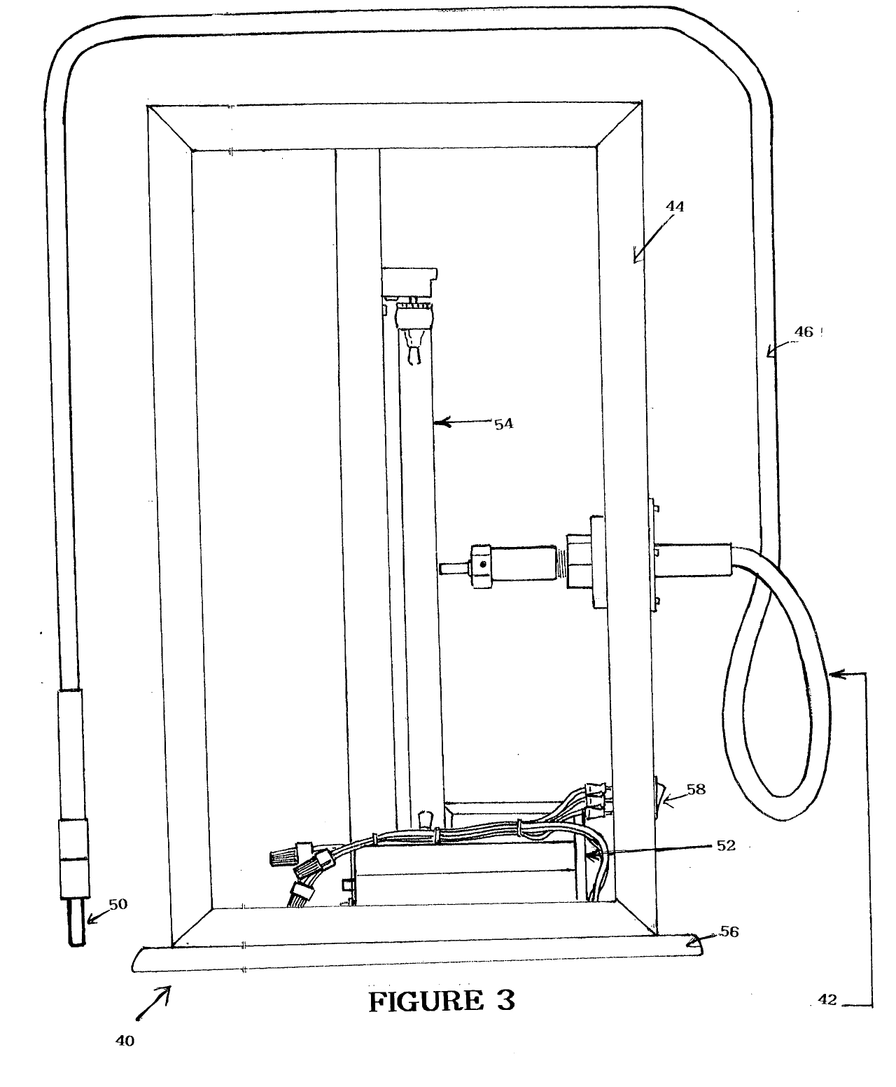

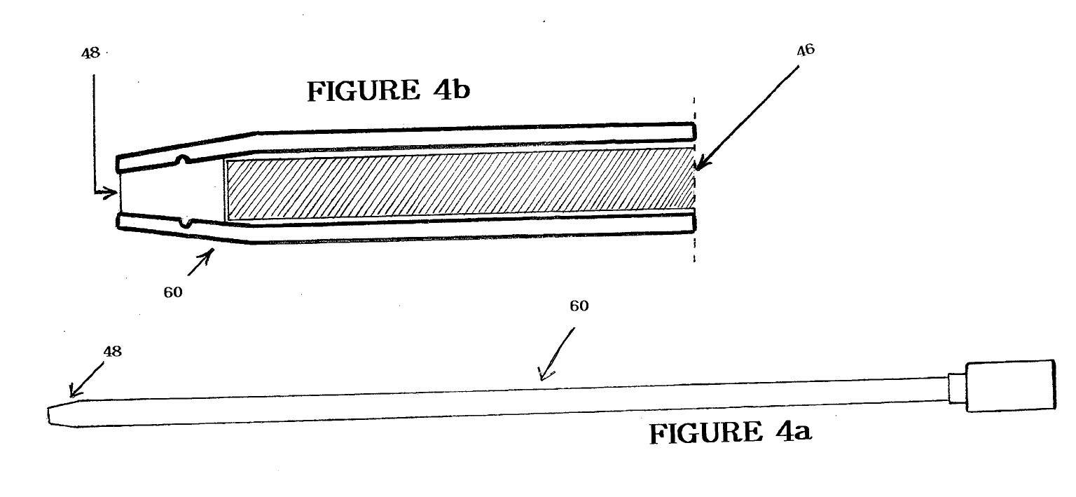

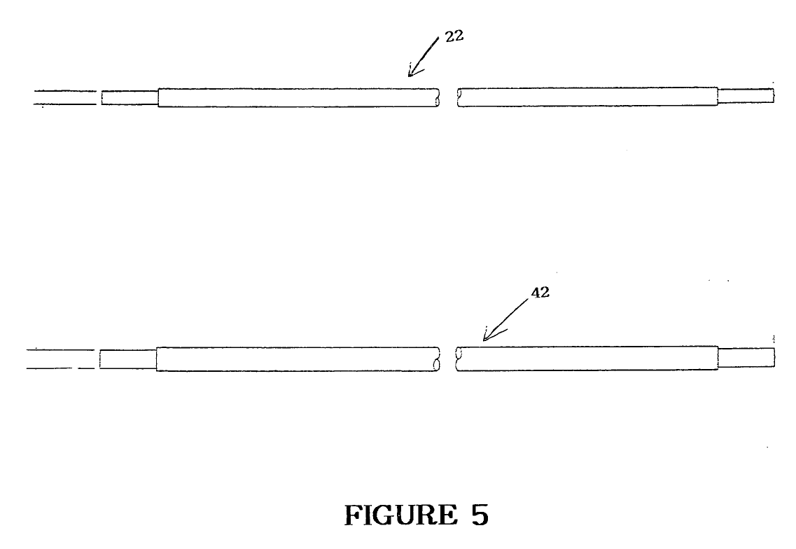

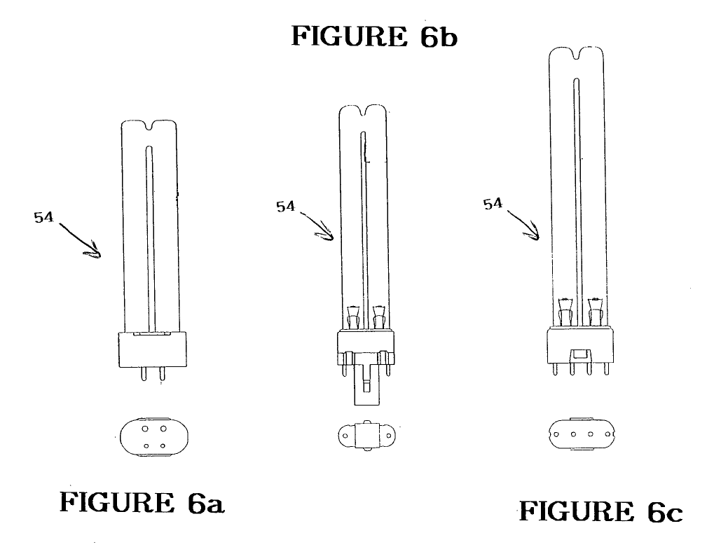

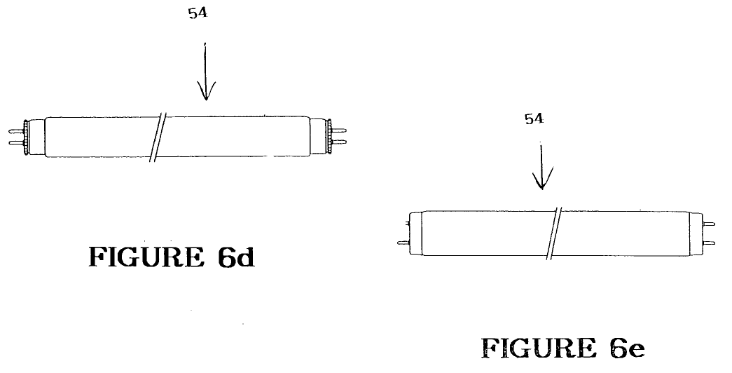

### DETAILED DESCRIPTION OF THE INVENTION

Ultraviolet light (UV) can is used to treat many diseases including infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Preferably, UV-C light is utilized in the present invention. UV-C light has a wavelength of 190 nm-100 nm.

The invention is a UV device 40 and a portable UV pack 20 that are connectable to a patient 10 via a port 12 to directly supply UV light to the patient's blood supply 14. Port 12 is surgically implanted in patient 10. Ports and catheters are well known in the art. They are for cancer patients receiving regular or continuous chemotherapy, diabetics and others. Alternatively, UV pack 20 could be surgically implanted under the patient's skin. In yet another alternative, a portion of UV pack 20 such as a portion of light guide 22 can be implanted in patient 10.

Portable UV pack 20 comprises housing 24, battery or other power source (not shown) and UV light source (not shown). Preferably, UV light source (not shown) is LEDs (not shown) emitting UV light at a therapeutic wavelength. More preferably, the UV light is UV-C light and ranges from 190 nm-100 nm. Alternatively, a plurality of LEDs, having one or more different wavelengths of UV light could be used. It is preferable that a substantial portion of the emitted light be UV-C.

In one embodiment, UV pack 20 has a light guide 22 made of rubber or other flexible tubing for housing one or more fiber optic strands 26. Alternatively, a liquid core light guide or other known light guide can be used. Emitted UV radiation travels to the end tip of the light guide 22 and is emitted. Emitted radiation directly illuminates patient's blood. Light guide 22 has transparent cover 28 at the end connectable to or insertable in the patient.

Light guide 22 has a connector 30 for coupling UV pack 20 to port 12 or catheter in the patient. Catheter may be inserted into port 12 or there may be connector 30 on one end of the flexible tube that mates with a connector on port 12.

In an alternative embodiment, light source such as a LED, or miniature light bulb is inserted through port 12 and directly illuminates the blood.

The UV pack 20 allows UV light to be supplied directly to the blood. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence.

Patient 10 can connect to UV pack 20 when a treatment is needed. Alternatively, UV pack 20 can remain connected to port 12 and be turned on only for treatment. UV pack 20 could be turned on and off manually. Alternatively, UV pack 20 could automatically turn off the light source after a set treatment time, such as 20 minutes. UV pack 20 could have a controller such as a computer or other smart interface that limits the number of treatments given time period, limits the total amount of treatment time in a given time period, automatically provides treatments, pulses the light source, or provides only particular wavelengths. The computer or other smart interface could keep a treatment record. The computer or other smart interface could communicate wirelessly, via the Internet or through other electronic means to automatically update the doctor's treatment records. Computer preferably can automatically adjust treatment time, wavelength or other factors based on patient input, doctor orders or other data.

In an alternative embodiment, UV pack 20 or a portion of UV pack 20, such as an end of light guide 22 is surgically attached to patient 10 or implanted in patient 10.

UV device 40 can be attached to patient 10 via port 12 to directly illuminate the blood. UV device 40 comprises housing 44, light guide 42, and UV light source 54. Preferably, UV light source 54 is a medical grade UV light bulb. UV light source 54 preferably emits light in the UV-C range. Housing 44 preferably has a weighted base 56. There are preferably electronics 52 such as a power supply or power cord for connection to a power source. UV device preferably has a manual on/off switch 58. Electronics 52 also preferably include a controller, a timer or smart interface such as a computer.

Catheter 60 with light guide 42 is inserted into port 12 to directly illuminate the blood. Light guide 42 may have connector 50 that mates with a connecter on port 12. Light guide 42 may be one or more fiber optic strands in a flexible tube. Alternatively, light guide 42 may be a liquid core light guide 46 or other known light guide. In yet another alternative, UV light source is a LED or small light bulb at the end of a flexible tube adapted to be inserted through port 12 to directly illuminate the blood.

UV device 40 allows UV light to be supplied directly to the blood. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence.

Patient 10 can connect to the UV device 40 when a treatment is needed. The UV device 40 could be turned on and off manually. Alternatively, UV device 40 could automatically turn off the light source after a set treatment time, such as 20 minutes. UV device 40 could have a controller, computer or other smart interface that limits the number of treatments given time period, limits the total amount of treatment time in a given time period, automatically provides treatments, pulses the LEDs, or provides only particular wavelengths if the UV pack has LEDs of various wavelengths. The computer or other smart interface could keep a treatment record. The computer or other smart interface could communicate wirelessly, via the Internet or through other electronic means to automatically update the doctor's treatment records. The computer could automatically adjust the treatment time based on input from the patient, the doctor, treatment records, or other data.

1. A UV blood illuminator comprising:
a housing;
a power supply;
a UV light source powered by said power supply; said UV light source in said housing and
a light guide, a first end of said light guide connected to a UV light source and a second end adapted for connection with a patient.

2. The UV blood illuminator of claim 1 further comprising at least one fiber optic strand in said light guide.

3. The UV blood illuminator of claim 2 wherein the UV light source emits UV-C radiation.

4. The UV blood illuminator of claim 3 wherein the UV light source comprises at least one LED.

5. The UV blood illuminator of claim 3 wherein the UV light source is a medical grade UV light bulb.

6. The UV blood illuminator of claim 2 further comprising a catheter at the second end and the at least one fiber optic strand is in said catheter.

7. The UV blood illuminator of claim 6 wherein a portion of the catheter adapted for insertion in a port is transparent.

8. The UV blood illuminator of claim 2 further comprising a connector at a second end, said connector adapted to mate with a port in a patient.

9. The UV blood illuminator of claim 2 wherein the second end is adapted to be surgically implanted in an artery or vein of a patient.

10. The UV blood illuminator of claim 9 wherein the blood illuminator is adapted to be surgically implanted in a patient.

11. The UV blood illuminator of claim 1 wherein the light guide is a liquid core light guide.

12. The UV blood illuminator of claim 11 wherein the UV light source emits UV-C radiation.

13. The UV blood illuminator of claim 11 wherein the UV light source comprises at least one LED.

14. The UV blood illuminator of claim 11 wherein the UV light source is a medical grade UV light bulb

15. The UV blood illuminator of claim 11 further comprising a catheter at the second end of said light guide.

16. The UV blood illuminator of claim 15 wherein a portion of the catheter adapted for insertion in a port is transparent.

17. The UV blood illuminator of claim 11 further comprising a connector at a second end, said connector adapted to mate with a port in a patient.

18. The blood illuminator of claim 1 further comprising a controller.

19. The blood illuminator of claim 18 wherein the controller is an on/off switch.

20. The blood illuminator of claim 18 wherein the controller automatically controls the light source.

21. The blood illuminator of claim 20 wherein the controller automatically controls the light source by pulsing the light, by automatically shutting off the light after a specified period of time, by automatically activating the light source at a specified time or by combinations thereof.

22. The UV blood illuminator of claim 18 wherein the controller is a computer.

23. The UV blood illuminator of claim 22 wherein the computer is adapted to maintain and/or transmit treatment records.

24. A UV blood illuminator comprising:
a housing,
a UV light source,
a power supply for supplying power to the UV light source,
a flexible tube adapted for insertion in a patient, a first end of said tube connected to said housing, and a second end housing said UV light source.

25. The UV blood illuminator of claim 24 wherein the UV light source emits UV-C radiation.

26. The UV blood illuminator of claim 25 wherein the UV light source comprises at least one LED.

27. The UV blood illuminator of claim 25 wherein the UV light source is a medical grade UV light bulb.

28. The UV blood illuminator of claim 25 further comprising a catheter at the second end and the UV light source is in said catheter.

29. The UV blood illuminator of claim 28 wherein a portion of the catheter adapted for insertion in a port is transparent.

30. The UV blood illuminator of claim 24 wherein the UV light source is adapted to be surgically implanted in an artery or vein of a patient.

31. The UV blood illuminator of claim 30 wherein the blood illuminator is adapted to be surgically implanted in a patient.

32. The blood illuminator of claim 24 further comprising a controller.

33. The blood illuminator of claim 32 wherein the controller is an on/off switch.

34. The blood illuminator of claim 32 wherein the controller automatically controls the light source.

35. The blood illuminator of claim 34 wherein the controller automatically controls the light source by pulsing the light, by automatically shutting off the light after a specified period of time, by automatically activating the light source at a specified time or by combinations thereof.

36. The UV blood illuminator of claim 32 wherein the controller is a computer.

37. The UV blood illuminator of claim 36 wherein the computer is adapted to maintain and/or transmit treatment records.

38. A blood illumination system comprising:
a blood illuminator and a patient, said blood illuminator comprising:
a housing, a power supply, a UV light source powered by said power supply, said UV light source in said housing and a light guide,
a first end of said light guide connected to a UV light source and a second end adapted for connection with the patient.

39. The blood illumination system of claim 38 wherein the patient has a port and the blood illuminator connects to the patient at the port.

40. The blood illumination system of claim 38 wherein the blood illuminator is surgically implanted in the patient.

41. A blood illumination system comprising:
a blood illuminator and a patient, said blood illuminator comprising:
a housing, a UV light source, a power supply for supplying power to the UV light source, a flexible tube adapted for connection with the patient, a first end of said tube connected to said housing, and a second end housing said UV light source.

42. The blood illumination system of claim 41 wherein the patient has a port and the blood illuminator connects to the patient at the port.

43. The blood illumination system of claim wherein the blood illuminator is surgically implanted in the patient.

44. A method of illuminating blood comprising:
connecting a blood illuminator directly with a patient's vein or artery,
illuminating the blood in the vein or artery for a period of time with UV light.

45. The method of claim 44 further comprising the steps of automatically turning the UV light on and off.

46. The method of claim 45 further comprising the steps of automatically recording treatment data.

47. The method of claim 46 further comprising the step of automatically providing the treatment data to a doctor.

48. The method of claim 45 further comprising the step of automatically adjusting the period which the UV light is on.
